(19) United States
(12) Patent Application Publication
(10) Pub. No.: US 2013/0226282 A1
(43) Pub. Date: Aug. 29, 2013

(54) STENT WIRES, AND METHOD FOR MANUFACTURING SUCH STENT WIRES AND STENTS

(71) Applicants: Sae-Young Ahn, Seoul (KR); Hyun-Hwa Kwon, Goyang-si (KR)

(72) Inventors: Sae-Young Ahn, Seoul (KR); Hyun-Hwa Kwon, Goyang-si (KR)

(73) Assignee: MEDISOURCEPLUS CO., LTD., Seoul (KR)

(21) Appl. No.: 13/882,260

(22) PCT Filed: Oct. 31, 2011
(86) PCT No.: PCT/KR11/08197

§ 371 Date: Apr. 29, 2013

(30) Foreign Application Priority Data
Oct. 29, 2010 (KR) 10-2010-0107248
Oct. 29, 2010 (KR) 10-2010-0107249

(51) Int. Cl. B21F 45/00; B22D 11/00; A61F 2/06

(52) U.S. Cl. CPC B21F 45/008; A61F 2/06; B22D 11/005
USPC 623/1.15; 29/592; 29/527.5; 140/71 R

(57) ABSTRACT

A method for manufacturing stent wires includes preparing three or more annular stent wires which has alternately arranged peaks and valleys, interconnecting the first and second stent wires, such that predetermined peaks of the second stent wire are caught in predetermined valleys of the first stent wire, passing a valley of a third stent wire below a free valley of the first stent wire and a free peak of the second stent wire, and interconnecting the third and second stent wires such that a peak of the third stent wire is caught in a valley of the second stent wire. The stent wires are connected in a stacked manner, thereby simplifying the manufacturing process, lengthening the lifespan of the stent wires, and improving the strength of the connection between two adjacent stent wires.

### TECHNICAL FIELD

The present invention relates to stent wires and a method of manufacturing such stent wires and stents, and more particularly, to stent wires which are manufactured by casting such that shapes are different according to portions, a method of manufacturing such stent wires by casting, and a method of manufacturing stents each of which is configured such that annular stent wires can be stacked on and connected to each other.

### BACKGROUND ART

In general, a blood vessel may have angiostenosis due to a blood clot, arteriosclerosis or so on, or an aneurysm in which a part of the blood vessel expands like a balloon due to aging or other diseases.

In a location of the blood vessel where angiostenosis or an aneurysm has occurred, a surgical operation is generally conducted. Replacement with an artificial blood vessel or bypass grafting is thereby performed in the corresponding location. Such a surgical operation has problems in that it leaves a large scar since a large incision has to be made in the diseased area, and that the effectiveness of the surgical operation is not so great. In addition, the same problems as those occur with the stricture of the throat, biliary stricture, the stricture of the urethra, and the blockage or stricture of other internal organs, as well as transjugular intrahepatic portosystemic shunt (TIPS).

For this reason, a variety of techniques for simply treating the above-described diseased area, such as the stricture of a body part or an aneurysm in a blood vessel, instead of performing the surgical operation has been recently disclosed. One of these techniques is treatment using a stent made of a shape memory metal. Stents are divided into non-vascular stents which are used in the throat or the internal organs and vascular stents which are used in blood vessels. Non-vascular stents are manufactured by the process of netting wires into a hollow cylindrical shape, since they have a predetermined minimum size. Vascular stents are manufactured by the process of cutting a base material using a laser, since it is difficult to machine vascular stents into a precise shape via the wire netting because of the very small size of vascular stents.

However, the manufacture of stents by the netting has drawbacks in that a separate netting jig is required, and that the complicated netting process makes the manufacturing difficult. In addition, in the case of manufacturing stents using the laser cutting technique, devices for the laser cutting as well as a very precise machining technique are necessarily required. This accordingly leads to the problem of the increased manufacturing cost.

In addition, since a stent wire is typically manufactured by drawing such that it has the shape of a straight line, it is required that both ends of a linear stent be connected in order to make a ring-shaped stent. However, when the both ends of the linear stent wire are connected to each other, there is a risk of damage to the internal organ or the blood vessel since the joint is not smooth. In addition, the process of connecting the stent wire is also required. This consequently makes the manufacturing process complicated and thus increases the manufacturing cost, which is problematic. In addition, when the stent wire has the joint as mentioned above, there is a problem in that the strength of the joint is weaker than that of the other portions, thereby reducing lifespan. It may be necessary to manufacture individual portions of the stent having various shapes depending on the environment or conditions where the stent is used. When the stent wire is manufactured by the drawing as mentioned above, there is a drawback in that the stent wire cannot be manufactured such that individual portions thereof have various sizes or shapes. Accordingly, a separate machining process is required in order to change the size or shape of each portion of the stent wire, thereby making the stent wire manufacturing process complicated and increasing the manufacturing cost, which is problematic.

It is, of course, possible to freely change the size or shape of each portion of a stent when manufacturing the stent by the laser cutting technique. However, the laser cutting devices are necessarily required and a very precise machining technique is required, thereby leading to the drawback of the increased manufacturing cost.

### DISCLOSURE

### Technical Problem

The present invention has been made to solve the foregoing problems with the prior art, and therefore an object of the present invention is to provide a method of manufacturing stents, which can simplify a manufacturing process and increase the lifespan of stent wires by interconnecting a plurality of stent wires in a stacking fashion while enhancing the strength of joints between two adjacent stent wires.

An object of the present invention is to provide a stent wire which is manufactured by casting such that its shape differs according to the portion. Also provided is a method of manufacturing stent wires, in which an annular stent wire can be manufactured by a single process, such that the size and shape of each portion of the stent wire can be variously formed without a separate machining process.

### Technical Solution

According to an aspect of the invention for realizing the foregoing object, provided is a method of manufacturing stents. The method includes: a first step of preparing at least three or more stent wires, each of which has an annular shape in a plan view, and includes peaks and valleys which alternate with each other; a second step of interconnecting the first and second stent wires such that predetermined peaks of the second stent wire are caught by predetermined valleys of the first stent wire, wherein the first and second stent wires are interconnected in a repeated pattern in which two consecutive peaks of the second stent wire are caught by two consecutive valleys of the first stent wire and one subsequent peak of the second stent wire is uncaught by one subsequent valley of the first stent wire; and a third step of connecting the third stent wire to the second stent wire by moving down valleys of the third stent wire to pass between the valleys of the first stent wire and the peaks of the second stent wire so that predetermined peaks of the third stent are caught by predetermined valleys of the second stent wire.

The second step may include causing the predetermined peaks of the second stent wire to be caught by the predetermined valleys of the first stent wire in a process of positioning the second stent wire above the first stent wire and then moving at least one of the first and second stent wires in the top-bottom direction.

The third step may include causing the peaks of the third stent to be caught by the valleys of the second stent wire in a process of positioning the third stent wire above the second stent wire and then moving down the third stent wire.

According to another aspect of the invention for realizing the foregoing object, provided is a method of manufacturing stents. The method includes: a first step of preparing at least three or more stent wires, each of which has an annular shape in a plan view, and includes peaks and valleys which alternate with each other; a second step of interconnecting the first and second stent wires such that predetermined peaks of the second stent wire are caught by predetermined valleys of the first stent wire, wherein the first and second stent wires are interconnected in a repeated pattern in which two consecutive peaks of the second stent wire are caught by two consecutive valleys of the first stent wire and one subsequent peak of the second stent wire is uncaught by one subsequent valley of the first stent wire; and a third step of connecting the third stent wire to the first stent wire by moving up peaks of the third stent wire to pass between the valleys of the first stent wire and the peaks of the second stent wire so that predetermined valleys of the third stent are caught by predetermined peaks of the first stent wire.

The second step may include causing the predetermined peaks of the second stent wire to be caught by the predetermined valleys of the first stent wire in a process of positioning the second stent wire above the first stent wire and then moving at least one of the first and second stent wires in a top-bottom direction.

The third step may include causing the peaks of the third stent to be caught by the valleys of the second stent wire in a process of positioning the third stent wire above the second stent wire and then moving down the third stent wire.

Each number of the peaks and the valleys may be set to a multiple of 3.

The first step may include manufacturing the stent wires by casting.

In each of the stent wires, the thickness of the peaks and the valleys may be greater than the thickness of remaining portions.

The first step may include a process of manufacturing the stent wires, each of which has a shape of a planar looped curve, and includes alternating outward and inward protrusions, and a process of bending each of the stent wires so that the outward protrusions are positioned above the inward protrusions, whereby the bent outward protrusions form the peaks, and the bent inward protrusions form the valleys.

The method may further include a fourth step of connecting the valley and the peak which are disposed at corresponding positions and are uncaught by each other.

The fourth step may include binding the valley and the peak using a separate connecting wire.

A fastening portion having a through-hole may be provided on one of the valley and the peak, which are disposed at corresponding positions and are uncaught by each other, and an extension that is insertable into the through-hole may be provided on the other one of the valley and the peak, which are disposed at corresponding positions and are uncaught by each other. The fourth step may include connecting the valley and the peak to each other by inserting the extension into the through-hole.

The extension may be connected to the fastening portion such that the extension is bent after being inserted into the through-hole. A gap is formed in the top-bottom direction in a connecting portion between the extension and the fastening portion.

According to a further aspect of the invention for realizing the foregoing object, provided is a stent wire that has peaks and valleys which alternate with each other. Each cross-sectional area of the peaks and the valleys is greater than a cross-sectional area of remaining portions.

A fastening portion having on through-hole may be formed in one of each of the valleys and each of the peaks. An extension which is configured so as to be insertable into the through-hole may be formed on the other one of each of the valleys and each of the peaks.

The length of the through-hole in a top-bottom direction may be greater than the thickness of the extension.

The stent wire may be manufactured by casting such that the planar shape thereof forms a looped curve.

According to a further aspect of the invention for realizing the foregoing object, provided is a method of manufacturing stent wires. The method includes: a first step of manufacturing a stent wire which has the shape of a planar looped curve and includes alternating outward and inward protrusions; and a second step of bending the stent wire so that the outward protrusions are positioned above the inward protrusions, whereby the bent outward protrusions form the peaks, and the bent inward protrusions form the valleys.

The first step may include manufacturing the stent wire by casting.

The first step may include forming each cross-sectional area of the outward and inward protrusions to be greater than the cross-sectional area of remaining portions.

The first step may include forming a fastening portion having a through-hole on one of each of the outward protrusions and each of the inward protrusions and an extension on the other one of each of the outward protrusions and each of the inward protrusions, the extension being configured so as to be insertable into the through-hole.

### Advantageous Effects

The method of manufacturing stents according to the present invention has the following advantages. It is possible to simplify a manufacturing process and increase the lifespan of stent wires by interconnecting a plurality of stent wires in a stacking fashion while enhancing the strength of joints between two adjacent stent wires. In addition, the stent wire according to the present invention has advantages in that the strength of the peaks and the valleys is enhanced, that coupling between stent wires is facilitated, and that the stent wire does not cause damage to the blood vessel or internal organism since it does not have a joint. Furthermore, the method of manufacturing stent wires according to the present invention has an advantage of being capable of increasing the efficiency of production of stent wires, since a separate machining process for forming the peaks and valleys is not required.

### DESCRIPTION OF DRAWINGS

FIG. 1 and FIG. 2 are perspective views showing a process of manufacturing a stent wire;

FIG. 3 and FIG. 4 are front elevation views showing a process of connecting two stent wires;

FIG. 5 is a front elevation view of a stent manufactured by a method of manufacturing stents according to the present invention;

FIG. 6 is a front elevation view showing a connection structure of a peak and a valley which are not caught by each other;

FIG. 7 is an exploded perspective view of a mold for manufacturing stent wires by casting;

FIG. 8 is a front elevation view of a second embodiment of the stent wire;

FIG. 9 is a front elevation view of a third embodiment of the stent wire;

FIG. 10 and FIG. 11 are front elevation and cross-sectional views showing the coupling structure of the third embodiment of the stent wire;

FIG. 12 and FIG. 13 are perspective and cross-sectional views showing a manufacturing process of a fourth embodiment of the stent wire.

### BEST MODE

Hereinafter a method of manufacturing stents according to an embodiment of the present invention will be described in detail with reference to the accompanying drawings.

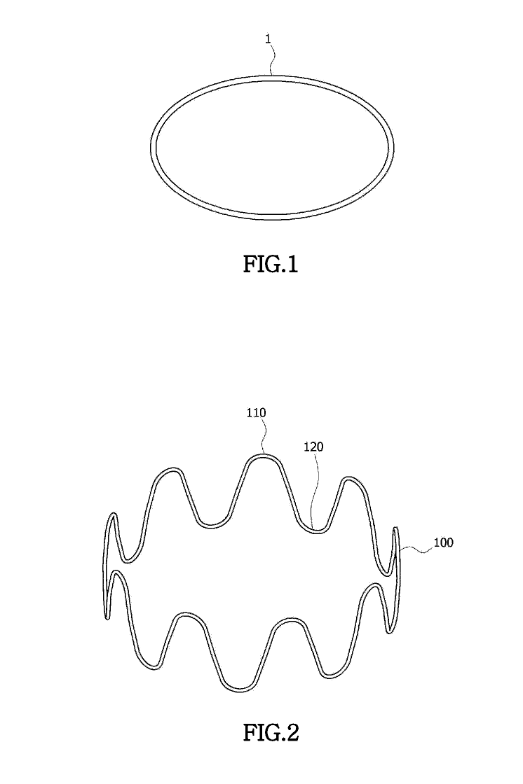

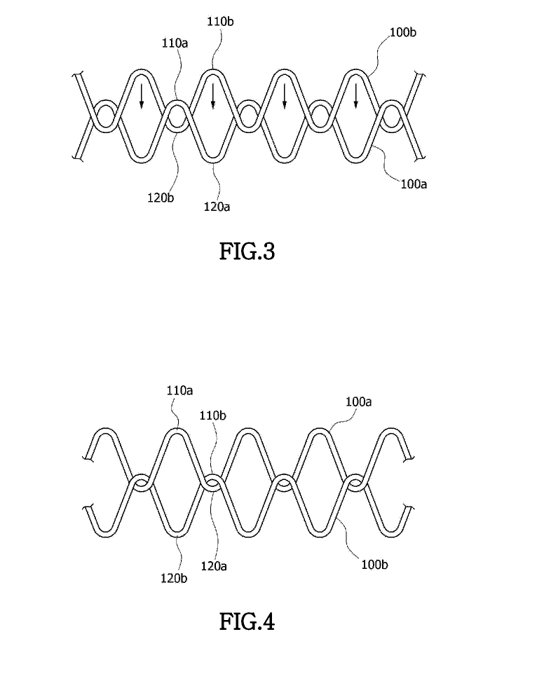

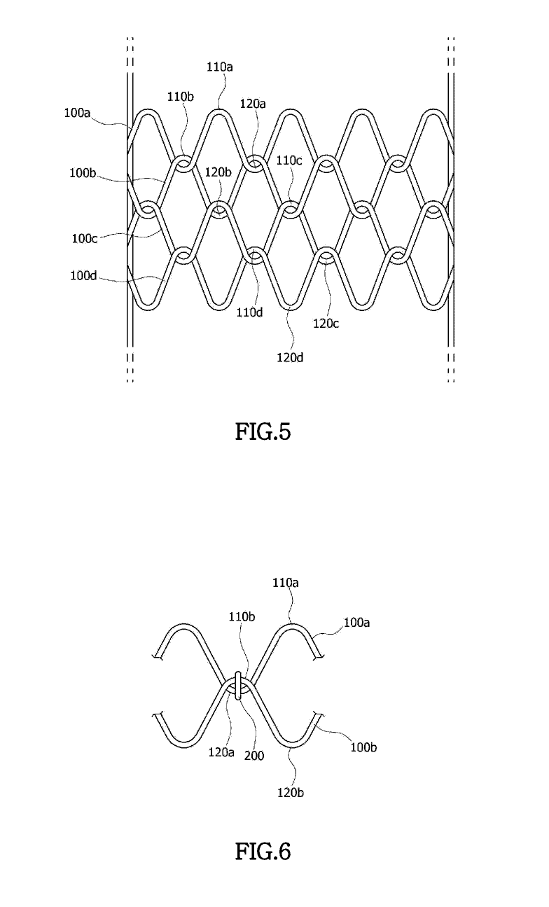

FIG. 1 and FIG. 2 are perspective views showing a process of manufacturing a stent wire, FIG. 3 and FIG. 4 are front elevation views showing a process of connecting two stent wires, and FIG. 5 is a front elevation view of a stent manufactured by a method of manufacturing stents according to the present invention.

The method of manufacturing stents according to the present invention is a method of manufacturing a cylindrical stent by connecting a plurality of annular stent wires 100 to each other, and includes a first step of preparing three or more stent wires 100, each of which has an annular shape in a plan view, and includes peaks 110 and valleys 120 which alternate with each other; a second step of interconnecting the first and second stent wires 100a and 100b such that the peaks 110 of the second stent wire 100b are caught by the valleys 120 of the first stent wire 100a; and a third step of interconnecting the second and third stent wires such that the peaks 110 of the third stent wire 100c are caught by the valleys 120 of the second stent wire 100b.

The stent wires 100 are typically manufactured by drawing such that they have the shape of a straight line. Therefore, preferably, the first step of preparing the stent wires 100 having the peaks 110 and the valleys 120 includes a process of manufacturing a ring-shaped base material 1, as shown in FIG. 1, and a process of forming the peaks 110 and the valleys 120, as shown in FIG. 2, by applying an upward pressing force to predetermined sections of the ring-shaped base material 1 and a downward pressing force to the other sections of the ring-shaped base material 1.

In addition, when interconnecting the first and second stent wires 100a and 100b such that the peaks 110b of the second stent wire 100b are caught by the valleys 120a of the first stent wire 100a as in the second step, as shown in FIG. 3, some portions of the second stent wire 100b are positioned at the inner diameter side of the first stent wire 100a, and the other portions of the second stent wire 100b are positioned at the outer diameter side of the first stent wire 100a. The section positioned at the inner diameter side of the first stent wire 100a and the section positioned at the outer diameter side of the first stent wire 100a are disposed such that they are repeated while alternating with each other. When the second stent wire 100b is moved down from the position shown in FIG. 3, the peaks 110b of the second stent wire 100b are caught to the valleys 120a of the first stent wire 100a. The second stent wire 100b is connected to the first stent wire 100a without moving downward further.

Here, when all of the corresponding portions of the first and second stent wires, i.e. the valleys 120a of the first stent wire 100a and the peaks 110b of the second stent wire 100b are caught by each other, a further annular stent wire 100, i.e. the third stent wire 100c can be connected to neither the first stent wire 100a nor the second stent wire 100b. The third stent wire 100c may, of course, be connected to the first or second stent wire 100a or 100b by cutting an intermediate portion of the third stent wire 100c and then binding the third stent wire 100c to the peaks 110a of the first stent wire 100a or the valleys 120b of the second stent wire 100b. In this case, however, there is a drawback in that the process of cutting and reconnecting the third stent wire 100c is required.

Therefore, the method of manufacturing stents according to the present invention is characterized in that the first and second stent wires 100a and 100b are not interconnected such that all of the peaks 110b of the second stent wire 100b are caught by the valleys 120a of the first stent wire 100a. Rather, predetermined peaks 110b of the second stent wire 100b are caught by the corresponding valleys 120a of the first stent wire 100a, but the other peaks 110b of the second stent wire 100b are uncaught by the other corresponding valleys 120a of the first stent wire 100a. As shown in FIG. 3, after the first, third and fourth valleys 120b of the second stent wire 100b are positioned in front of the first stent wire 100a and the second and fifth valleys 120b of the second stent wire 100b are positioned behind the first stent wire 100a, the second stent wire 100b is moved down. Consequently, as shown in FIG. 4, the first, second and fourth peaks 110b of the second stent wire 100b are caught on the valleys 120a of the first stent wire 100a, and the third peak 110b of the second stent wire 100b is uncaught by the valley 120a of the first stent wire.

When the other peaks 110b of the second stent wire 100b are uncaught by the other valleys 120a of the first stent wire 100a, a worker can insert the valleys 120c of the third stent wire 100c between the valleys 120a of the first stent wire 100a and the peaks 110b of the second stent wire 100b (the third valley 120a of the first stent wire 100a and the third peak 110b of the second stent wire 100b in FIG. 4) which are uncaught by each other, and then move down the third stent wire 100c, so that the peaks 110c of the third stent wire 100c are caught by the valleys 120b of the second stent wire 100b, as shown in FIG. 5.

Here, if the peaks 110b of the second stent wire 100b caught by the valleys 120a of the first stent wire 100a and the peaks 110b of the second stent wire 100b uncaught by the valleys 120a of the first stent wire 100a are repeated in a ratio of 2:1, when the third stent wire 100c is connected to the second stent wire 100b by inserting the valleys 120c of the third stent wire 100c between the valleys 120a of the first stent wire 100a and the peaks 110b of the second stent wire 100b and then moving down the third stent wire 100c as described above, all of the peaks 110c of the third stent wire 100c are not caught on the valleys 120b of the second stent wire 100b but only predetermined peaks 110c of the third stent wire 100c are caught on the corresponding valleys 120b of the second stent wire 100b. As shown in FIG. 5, the first, third and fourth peaks 110c of the third stent wire 100c are connected to the corresponding valleys 120b of the second stent wire 100b. Accordingly, the worker can connect a fourth stent wire 100d to the third stent wire 100c by inserting valleys 120d of the fourth stent wire 100d between the second and fifth valleys 120b of the second stent wire 100b and the second and fifth peaks 110c of the third stent wire 100c and then moving down the fourth stent wire 100d.

It is, of course, possible to connect the third stent wire 100c to the second stent wire 100b and the fourth stent wire 100d to the third stent wire 100c when the ratio of repeating the peaks 110b of the second stent wire 100b caught by the valleys 120a of the first stent wire 100a and the peaks 110b of the second stent wire 100b uncaught by the valleys 120a of the first stent wire 100a is 3:1 or 2:2 instead of 2:1. In this case, however, there is a risk of damage to connected portions between the stent wires 100 when an external force is applied, since the number of the connected portions between the stents 100 is decreased. Therefore, it is preferable that the adjacent stent wires are interconnected in a repeated pattern in which first two consecutive peaks 110 of one stent wire are caught by first two consecutive valleys 120 of the adjacent stent wire and then one subsequent peak 110 of one stent wire is uncaught by one subsequent valley 120 of the adjacent stent wire, as illustrated in this embodiment. Here, it is preferred that the number of the peaks 110 and the number of the valleys 120 be set to multiples of 3, such that the peaks 110 caught by the valleys 120 and the peaks 110 uncaught by the valleys 120 are repeated in a ratio of 2:1.

In addition, although only the method of connecting two stent wires to each other by the operation of moving down one stent wire 100 that is to be connected has been illustrated in this embodiment, the two stent wires 100 can be connected to each other by the operation of moving up one stent wire 100 that is to be connected. For instance, it is possible to connect the second stent wire 100b to the first stent wire 100a by positioning the second stent wire 100b below the first stent wire 100a and then moving up the second stent wire 100b such that the valleys 120b of the second stent wire 100b are caught by the peaks 110a of the first stent wire 100a, and connect the third stent wire 100c to the second stent wire 100b by inserting the peaks 110c of the third stent wire 100c between the valleys 120a of the first stent wire 100a and the peaks 110b of the second stent wire 100b, which are uncaught by each other, and then moving up the third stent wire 100c such that the valleys 120c of the third stent wire 100c are caught by the peaks 110b of the second stent wire 100b. A detailed description of this method of interconnecting the stent wires 100 by moving up the additional stent wire 100 will be omitted, since the principle and structure thereof for interconnecting the two stent wires are identical to those of the above-described method of interconnecting the stent wires 100 by moving down the additional stent wire 100 except for the direction in which the stent wires are connected.

FIG. 6 is a front elevation view showing a connection structure of a peak and a valley which are not caught by each other.

In the configuration in which some valleys 120 are uncaught by corresponding peaks 110 as shown in FIG. 5, when tension is applied in the lengthwise direction of the stent (the up-down direction in FIG. 5), the tension is concentrated on the remaining valleys 120 and peaks 110, each valley 120 being connected to a corresponding peak 110. Consequently, the strength of the stent to the tension may be decreased. In order to overcome this problem, the method of manufacturing stents according to the present invention can also include a fourth step of interconnecting the valley 120a and the peak 110b which are disposed at corresponding positions and uncaught by each other, as shown in FIG. 6.

In this case, welding, brazing or the like can be applied as a technique for interconnecting the valley 120a and the peak 110b which are uncaught by each other. However, since the stent wires 100 must be heated for the welding or brazing, the structure of the stent wire 100 may be deformed, thereby causing a problem of an unexpected decrease in the strength. Therefore, it is preferred that the fourth step be applied to interconnect the valley 120a and the peak 110b which are uncaught by each other by binding the valley 120a and the peak 110b together using a separate connecting wire 200, as shown in FIG. 6. A description of the technique of interconnecting the two stent wires 100 by binding the valley 120 and the peak 110 of the different stent wires using the separate connecting wire 200 will be omitted, since this technique is widely used in the manufacturing field of stents.

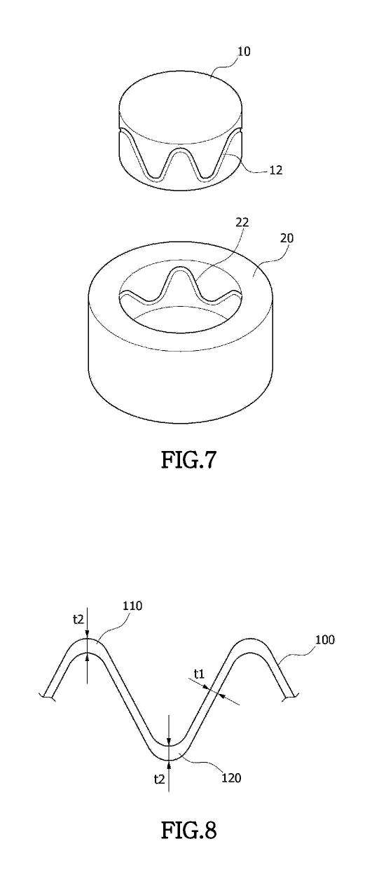

FIG. 7 is an exploded perspective view of a mold for manufacturing stent wires by casting, and FIG. 8 is a front elevation view of a second embodiment of the stent wire.

When the stent wires 100 are interconnected such that the peaks 110 are selectively caught by the valleys 120 of another stent wire as described above, the peaks 110 and the valleys 120 may be damaged first. This is because, when tension is applied in the lengthwise direction of the stent, the tension is concentrated on the peaks 110 and the valleys 120. Therefore, it is preferable to increase the thickness of the valleys 120 and the peaks 110 in order to improve the endurance of a stent against tension. Since the stent wires 100 are generally manufactured by drawing, the problem is that it is very difficult to make only the valleys 120 and the peaks 110 thick. In addition, in the case of manufacturing the stent wires 100 by drawing, the problem is that it is impossible to manufacture an annular stent wire 100 without a joint.

Therefore, it is preferred that the stent wire 100 according to the present invention be manufactured by casting such that the peaks 110 and the valleys 120 are thicker than the other portions. Specifically, the method of manufacturing stents according to the present invention can manufacture the annular stent wire 100 without a joint using a cylindrical inner mold 10 and an outer mold 20, as shown in FIG. 7. The inner mold 10 has an outer groove 12 in the outer circumference thereof. The outer mold 20 has a hollow cylindrical inner space, into which the inner mold 10 can be fitted, and an inner groove 22 in the inner circumference thereof. Since the outer groove 12 and the inner groove 22 form a passage for molten metal having a circular cross-section when they are joined to each other, the worker can manufacture the stent wire 100 without a joint by pouring molten metal into the passage for molten metal defined by the outer and inner grooves 12 and 22 after fitting the inner mold 10 into the inner space of the outer mold 20 so that the outer groove 12 is aligned with the inner groove 22.

In addition, as shown in FIG. 7, when the outer and inner grooves 12 and 22 are made into the form of waves having a predetermined period, the stent wire 100 having the peaks 110 and the valleys 120 is manufactured. Accordingly, the separate machining process for forming the peaks 110 and the valleys 120 becomes unnecessary, thereby leading to the effects of improved productivity and reduced manufacturing cost.

In addition, the manufacture of the stent wire 100 by casting is advantageous in that it is possible to easily produce the stent wire 100, in which the thickness t₂ of the peaks 110 and the valleys 120 is greater than the thickness t₁ of the other portions, by only the operation of forming the top curvature portions of the outer and inner grooves 12 and 22 that are supposed to form the peaks 110 and the bottom curvature portions of the outer and inner grooves 12 and 22 that are supposed to form the valleys 120 such that the inner diameter thereof is greater than that of the other portions. Although only the method of manufacturing the stent wire 100 having the thicker peaks and valleys 110 and 120 has been illustrated in this embodiment, it is of course possible to increase the cross-sectional area of the valleys 120 and the peaks 110 by forming the stent wire 100 by drawing and then adding a reinforcement material to the valleys 120 and the peaks 110.

In addition, when the stent wire 100 is manufactured by casting as described above, the separate machining process for forming the peaks 110 and the valleys 120 becomes unnecessary, thereby advantageously improving the productivity of the stent wire 100.

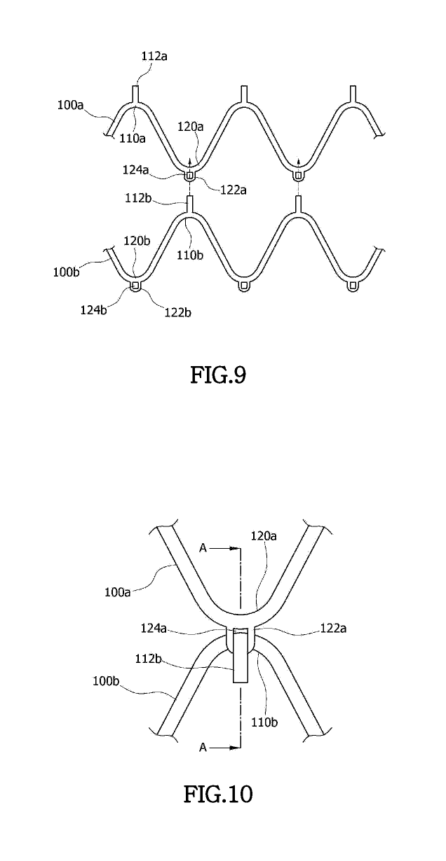

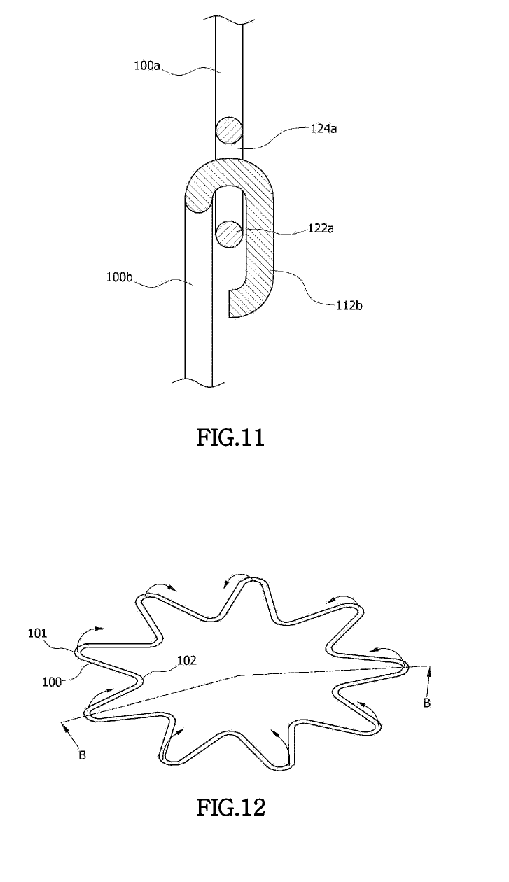

FIG. 9 is a front elevation view of a third embodiment of the stent wire, and FIG. 10 and FIG. 11 are front elevation and cross-sectional views showing the coupling structure of the third embodiment of the stent wire.

When interconnecting the valleys 120 and the peaks 110 which are disposed at corresponding positions but are uncaught by each other, the valleys 120 and the peaks 110 can be connected to each other using the separate connecting wire 200, as shown in FIG. 6. In this case, however, the difficult operation decreases productivity, which is problematic. Therefore, the stent wire 100 according to the present invention can have fastening means in the valleys 120 and the peaks 110 such that the stent wire 100 can be connected to another stent wire 100 a separate connecting wire.

Specifically, as shown in FIG. 9, fastening portions 122a each having a through-hole 124a are provided on the valleys 120a of the first stent wire 100a, and extensions 112b that can be inserted into the through-holes 124a are formed on the peaks 110b of the second stent 100b. The valleys 120 and the peaks 110 can be connected to each other by inserting the extensions 112b into the through-holes 124a. Here, it is preferred that the extensions 112b be bent after being inserted into the through-holes 124b, as shown in FIG. 10 and FIG. 11, such that the extensions 112b inserted into the through-holes 124b are not dislodged from the through-holes 124b.

In addition, when the valleys 120a of the first stent wire 100a and the peaks 110b of the second stent wire 100b are coupled to each other, the distance between each valley 120a of the first stent wire 100a and the counterpart peak 110b of the second stent wire 100b is required to be variable so that the length of the stent can vary within a predetermined range when tension is applied to the stent. When the extension 112b is fixedly coupled to the fastening portion 122a, the distance between the valley 120a of the first stent wire 100a and the peak 110b of the second stent wire 100b is not changeable, which is problematic. Therefore, when the extension 112b inserted into the through-hole 124a is bent, it is preferred that a gap be formed in the top-bottom direction in the connecting portion between the extension 112b and the fastening portion 122a. That is, as shown in FIG. 11, it is preferred that the top-bottom width of the through-hole 124a be formed greater than the thickness of the extension 112b, such that the extension 112b inserted into the through-hole 124a can move in the top-bottom direction. In addition, extensions 112a can also be formed on the peaks 110a of the first stent wire 100a and fastening portions 122b can also be formed on the valleys 120b of the second stent wire 100b, such that the first stent wire 100a and the second stent wire 100b can be connected with a third stent wire 100.

In the meantime, the fastening portions 122a and 122b and the extensions 112a and 112b may be added to the stent wire 100 which is manufactured by drawing. In this case, however, the process of manufacturing the fastening portions 122a and 122b and the extensions 112a and 112b and the process of mounting the fastening portions 122a and 122b and the extensions 112a and 112b to the valleys 120 and the peaks 110 are additionally required. This makes the manufacturing process complicated and increases the manufacturing cost, which is disadvantageous. Therefore, when intending to manufacture the stent wires having the fastening portions 122a and 122b and the extensions 112a and 112b, the use of casting is preferable. In addition, the extensions 112a can also be formed on the peaks 110a of the first stent wire 100a and the fastening portions 122b can also be formed on the valleys 120b of the second stent wire 100b, such that the first stent wire 100a and the second stent wire 100b can be connected with a third stent wire 100. Although only the structure in which the fastening portions 122 protrude downward from the bottom of the valleys 120 has been illustrated in this embodiment, the direction in which the fastening portions 122 protrude can be changed into a variety of directions, such as the upward or downward direction.

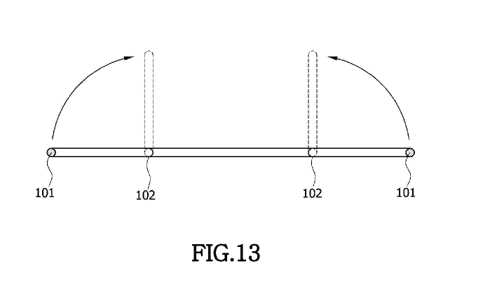

FIG. 12 and FIG. 13 are perspective and cross-sectional views showing a manufacturing process of a fourth embodiment of the stent wire.

The process of manufacturing the stent wire 100 which possesses the peaks 110 and the valleys 120 using the inner mold 10 and the outer mold 20, as shown in FIG. 7, has the advantage in that the manufacture of the stent wire 100 becomes simple since no separate processes for forming the peaks 110 and the valleys 120 are required. However, it may be difficult to fabricate the molds since the dimensions of the inner mold 10 and the outer mold 20 must be accurately managed in order to manufacture the three-dimensional stent wire 100.

Therefore, the method of manufacturing stent wires according to the present invention can be devised such that it forms the peaks 110 and the valleys 120 by a bending process after manufacturing the stent wire 100 having a two-dimensional shape, i.e. a planar shape. Specifically, the method of manufacturing stent wires according to the present invention includes a first step of preparing a stent wire having the shape of a planar looped curve, which includes alternating outward and inward protrusions 101 and 102, as shown in FIG. 12, and a second step of bending the wire stent so that the outward protrusions 101 are positioned above the inward protrusions 102, as shown in FIG. 13. When the wire stent is bent so that the outward protrusions 101 are positioned above the inward protrusions 102, the stent wire 100 has the cylindrical shape shown in FIG. 2, in which the outward protrusions 101 form the peaks 110 and the inward protrusions 102 form the valleys 120.

Although the stent wire 100 having the planar looped curve shown in FIG. 12 requires a separate mold since it must be manufactured by casting, the structure of the mold for casting a two-dimensional product is much simpler than the structure of molds for casting a three-dimensional product. The advantage is that the manufacture of the mold becomes much easier. Specifically, when the stent wire 100 is manufactured by the process shown in FIGS. 12 and 13, the peaks 110 and the valleys 120 are formed by only the operation of bending the outward protrusions 101 to 90° about the inward protrusions 102. The manufacture of the stent wire 100 becomes simpler than in the process shown in FIG. 1 and FIG. 2, and the fabrication of the mold becomes easier than in the process shown in FIG. 7.

In addition, as shown in FIGS. 12 and 13, when the peaks 110 and the valleys 120 are formed by bending the stent wire 100 having the shape of a planar looped curve, it is also possible to manufacture the stent wire 100, in which the cross-sectional area of the peaks 110 and the valleys 120 is greater than the cross-sectional area of the other portions, by forming the cross-section area of the outward protrusions 101 and the inward protrusions 102 to be greater than the cross-section area of the other portions. In addition, it is possible to facilitate connection between different stent wires 100 by forming the fastening portions 122a and 122b having the through-holes 124a and 124b (see FIG. 9) on the outward protrusions 101 or the inward protrusions 102 and forming the extensions 110a and 110b, which are configured so as to be inserted into the through-holes 124a and 124b, on the inward protrusions 102 or the outward protrusions 10. Descriptions of the effects obtained from the cross-sectional area of the peaks 110 and the valleys 120 being greater than the cross-sectional area of the other portions and the effects obtained from the formation of the fastening portions 122a and 122b and the extensions 110a and 110b will be omitted, since they have been described in detail with reference to the embodiment shown in FIG. 8.

While the present invention has been described in detail with reference to the certain exemplary embodiments, the scope of the present invention is not limited to the certain embodiments but shall be construed by the appended claims. In addition, it will be understood by a person having ordinary skill in the art that various modifications and variations can be made without departing from the scope of the present invention.

1. A method of manufacturing stents, comprising:
a first step of preparing at least three or more stent wires, each of which has an annular shape in a plan view, and includes peaks and valleys which alternate with each other;
a second step of interconnecting the first and second stent wires such that predetermined peaks of the second stent wire are caught by predetermined valleys of the first stent wire, wherein the first and second stent wires are interconnected in a repeated pattern in which two consecutive peaks of the second stent wire are caught by two consecutive valleys of the first stent wire and one subsequent peak of the second stent wire is uncaught by one subsequent valley of the first stent wire; and
a third step of connecting the third stent wire to the second stent wire by moving down valleys of the third stent wire to pass between the valleys of the first stent wire and the peaks of the second stent wire so that predetermined peaks of the third stent are caught by predetermined valleys of the second stent wire.

2. The method according to claim 1, wherein the second step comprises causing the predetermined peaks of the second stent wire to be caught by the predetermined valleys of the first stent wire in a process of positioning the second stent wire above the first stent wire and then moving at least one of the first and second stent wires in a top-bottom direction.

3. The method according to claim 1, wherein the third step comprises causing the peaks of the third stent to be caught by the valleys of the second stent wire in a process of positioning the third stent wire above the second stent wire and then moving down the third stent wire.

4. (canceled)

5. The method according to claim 1, wherein the second step comprises causing the predetermined peaks of the second stent wire to be caught by the predetermined valleys of the first stent wire in a process of positioning the second stent wire above the first stent wire and then moving at least one of the first and second stent wires in a top-bottom direction.

6. The method according to claim 1, wherein the third step comprises causing the peaks of the third stent to be caught by the valleys of the second stent wire in a process of positioning the third stent wire above the second stent wire and then moving down the third stent wire.

7. The method according to claim 1, wherein each number of the peaks and the valleys is a multiple of 3.

8. The method according to claim 1, wherein the first step comprises manufacturing the stent wires by casting.

9. The method according to claim 8, wherein, in each of the stent wires, each thickness of the peaks and the valleys is greater than a thickness of remaining portions.

10. The method according to claim 8, wherein the first step comprises a process of manufacturing the stent wires, each of which has a shape of a planar looped curve, and includes alternating outward and inward protrusions, and a process of bending each of the stent wires so that the outward protrusions are positioned above the inward protrusions, whereby the bent outward protrusions form the peaks, and the bent inward protrusions form the valleys.

11. The method according to claim 1, further comprising a fourth step of connecting the valley and the peak which are disposed at corresponding positions and uncaught by each other.

12. The method according to claim 11, wherein the fourth step comprising binding the valley and the peak using a separate connecting wire.

13. The method according to claim 11, wherein a fastening portion having a through-hole is provided on one of the valley and the peak, which are disposed at corresponding positions and uncaught by each other, and an extension that is insertable into the through-hole is provided on the other one of the valley and the peak, which are disposed at corresponding positions and uncaught by each other, wherein the fourth step comprises connecting the valley and the peak to each other by inserting the extension into the through-hole.

14. The method according to claim 13, wherein the extension is connected to the fastening portion such that the extension is bent after being inserted into the through-hole, a gap being formed in a top-bottom direction in a connecting portion between the extension and the fastening portion.

15. A stent wire comprising peaks and valleys which alternate with each other, each cross-sectional area of the peaks and the valleys being greater than a cross-sectional area of remaining portions.

16. A stent wire comprising peaks and valleys which alternate with each other, wherein a fastening portion having on through-hole is formed in one of each of the valleys and each of the peaks, and an extension which is configured so as to be insertable into the through-hole is formed on the other one of each of the valleys and each of the peaks.

17. The stent wire according to claim 16, wherein the extension is flexible so as to be bent after being inserted into the through-hole.

18. The stent wire according to claim 17, wherein a length of the through-hole in a top-bottom direction is greater than a thickness of the extension.

19. The stent wire according to claim 15, wherein the stent wire is manufactured by casting such that a planar shape thereof forms a looped curve.

20. A method of manufacturing stent wires, comprising:
a first step of manufacturing a stent wire which has a shape of a planar looped curve and includes alternating outward and inward protrusions; and
a second step of bending the stent wire so that the outward protrusions are positioned above the inward protrusions, whereby the bent outward protrusions form the peaks, and the bent inward protrusions form the valleys.

21. The method according to claim 20, wherein the first step comprises manufacturing the stent wire having the shape of a planar looped curve by casting.

22. The method according to claim 20, wherein the first step comprises forming each cross-sectional area of the outward and inward protrusions to be greater than a cross-sectional area of remaining portions.

23. The method according to claim 20, wherein the first step comprises forming a fastening portion having a through-hole on one of each of the outward protrusions and each of the inward protrusions and an extension on the other one of each of the outward protrusions and each of the inward protrusions, the extension being configured so as to be insertable into the through-hole.
